(19) United States
(12) Patent Application Publication
(10) Pub. No.: US 2024/0261148 A1
(43) Pub. Date: Aug. 8, 2024

(54) TREATMENT APPARATUS WITH AN OPHTHALMOLOGICAL LASER AND AT LEAST TWO FOCUS ADJUSTING DEVICES

(71) Applicant: SCHWIND EYE-TECH-SOLUTIONS GMBH, Kleinostheim (DE)

(72) Inventors: Samuel ARBA MOSQUERA, Aschaffenburg (DE); Luise KRÜGER, Aschaffenburg (DE); Mario SHRAIKI, Ober-Ramstadt (DE)

(21) Appl. No.: 18/433,965

(22) Filed: Feb. 6, 2024

(30) Foreign Application Priority Data
Feb. 8, 2023 (DE) 10 2023 103 034.5

(51) Int. Cl. A61F 9/008; A61B 18/20

(52) U.S. Cl. CPC A61F 9/00804; A61B 2018/20553; A61F 2009/00872

(57) ABSTRACT

A treatment apparatus with at least one ophthalmological laser is disclosed, wherein the treatment apparatus includes at least two focus adjusting devices, which are formed to adjust a focus position of a laser beam of the laser in a depth direction, wherein the first focus adjusting device is formed to adjust the focus point over a first depth distance range with a first speed, and wherein the second focus adjusting device is formed to adjust the focus point over a second depth distance range with a second speed, wherein the first depth distance range is larger than the second depth distance range and the first speed is smaller than the second speed.

### FIELD

The invention relates to a treatment apparatus, which includes at least one ophthalmological laser and at least two focus adjusting devices.

### BACKGROUND

Treatment apparatuses and methods for controlling ophthalmological lasers for correcting a visual disorder and/or pathologically or unnaturally altered areas of the cornea are known in the prior art. Therein, pulsed lasers and a focus adjusting device or beam focusing device can for example be formed such that laser pulses effect a photodisruption and/or photoablation in a focus situated within the organic tissue to remove a tissue, in particular a tissue lenticule, from the cornea.

Herein, a beam deflection device of the treatment apparatus can for example deflect a focus point in x/y directions or radial directions and a focus adjusting device can adjust the focus point in z-direction or depth direction. If an area to be irradiated in the eye, in particular in the cornea, is asymmetric, the control in depth direction can be aggravated since these settings can often be performed slowly and/or with a low adjustment distance.

### SUMMARY

Therefore, the object of the invention is in providing a treatment apparatus, which has an improved setting of a focus point.

This object is solved by the devices according to the invention. Advantageous embodiments of the invention are disclosed in the respective dependent claims, the following description as well as the figures.

The invention is based on the idea that two focus adjusting devices for the treatment apparatus are provided, which are each formed for different requirements. Preferably, one focus adjusting device can slowly adjust the focus point in depth direction, but with much stroke, and the other focus adjusting device can fast adjust the focus point, but with less stroke.

An aspect of the invention relates to a treatment apparatus with at least one ophthalmological laser, wherein the treatment apparatus includes at least two focus adjusting devices, which are formed to adjust a focus position of a laser beam of the laser in a depth direction, wherein a first focus adjusting device is formed to adjust the focus point over a first depth distance range with a first speed, and wherein the second focus adjusting device is formed to adjust the focus point over a second depth distance range with a second speed, wherein the first depth distance range is larger than the second depth distance range and the first speed is smaller than the second speed.

Thus, the first focus adjusting device has much stroke, but is slow, and the second focus adjusting device has little stroke, but is fast. In particular the first focus adjusting device can have an inferior resolution compared to the second focus adjusting device. In other words, a coarse setting of the focus position in depth direction can be achieved by the first focus adjusting device, wherein fine settings can be performed with the second, faster focus adjusting device.

By the depth distance range, the path in z-direction is meant, which the respective focus adjusting device can travel. By the speed, a path length per time is meant, with which the focus point can be adjusted in z-direction by the respective focus adjusting device.

By the invention, the advantage arises that different irradiation patterns, in particular asymmetric irradiation patterns, can be generated in improved manner since an extensive field of application can be covered by the focus adjusting devices. Preferably, the configuration with two focus adjusting devices can also be provided for other apparatuses such as for example 3D microscopy or confocal microscopy.

The invention also includes embodiments, by which additional advantages arise.

In an embodiment the second focus adjusting device is integrated in the first focus adjusting device, and wherein the first focus adjusting device is formed to also adapt the focus point of the second focus adjusting device upon adjusting the first focus adjusting device in depth direction. This means that if the first focus adjusting device is moved, the second focus adjusting device is also moved with the same path distance at the same time because it is integrated in the first focus adjusting device. Thus, a coarse setting or orientation can be performed with the first focus adjusting device and a fine tuning via the second focus adjusting device. In this context, integrated means that the focus position of the respective focus adjusting device is dependent on each other. Herein, it can for example be provided that the second focus adjusting device is formed as a lens or lens system, wherein the focus point can be shifted by adjusting at least one lens. Therein, this one or these multiple lenses of the second focus adjusting device can be located in the first focus adjusting device, which is formed as a carrier device, for example a type of box, which can be adjusted along the z-direction. Hereby, a preferred configuration of the treatment apparatus can be achieved.

In a further embodiment the first and the second focus adjusting device are arranged in series. Thus, a laser beam of the laser can for example first pass through the first focus adjusting device and subsequently through the second focus adjusting device. Preferably, it can be provided that the second, fast focus adjusting device is first arranged based on the laser and the first, slow focus adjusting device thereafter. Therein, the focus adjusting devices can comprise one or more lenses, for example in the form of an objective, wherein at least one lens can respectively be shifted in propagation direction of the laser. Thus, two lenses can for example be provided, which can have a similar refractive power and diameter, wherein one of the lenses moves in relation to the other one to set the focus in z-direction. Alternatively, both lenses can also shift to each other. Particularly preferably, it can be provided that the lenses can also transversally shift to each other in addition to an axial shift, that is in laser propagation direction, such as for example Alvarez or Lohmann lenses. Hereby, a further preferred configuration of the treatment apparatus can be provided.

In a further embodiment the first and the second focus adjusting device are parallel arranged in the treatment apparatus, wherein the treatment apparatus comprises a switching element, which is formed to selectively adjust the laser beam via the first or the second focus adjusting device. For example, a movable mirror can be used as the switching element, which can deflect the laser beam via the first or second focus adjusting device depending on a control. Thus, according to required adjustment path or speed, the suitable focus adjusting device can be controlled. Particularly preferably, it is provided that the focus adjusting device, via which the laser beam is currently not directed, is already moved into an intended treatment position, before the laser is changed to this focus adjusting device by the switching element. Hereby, a further preferred form of configuration of the treatment apparatus can be provided.

In a further embodiment the first depth distance range has an adjustment distance of 500 μm to 5 mm, preferably of 2 to 3 mm. In other words, the first focus adjusting device can be formed to adjust the focus point in a range, which is at least 500 μm or maximally 5 mm large.

In a further embodiment the first speed includes 3 mm/s to 850 mm/s, in particular 50 to 100 mm/s. This means that the first focus adjusting device can move the focus point with a speed, which is between 3 mm/s and 850 mm/s, in depth direction. These speeds can be an average speed, wherein a maximum speed can preferably have 6 mm/s to 1700 mm/s.

In a further embodiment a resolution of the first focus adjusting device has 100 nm to 1.5 μm, preferably 400 nm. This means that the first focus adjusting device can set the focus point accurately up to 1.5 μm, preferably up to 100 nm. Therein, response times can for example be in a range from 1 ms to 400 ms, in particular between 15 and 30 ms.

In a further embodiment the second depth distance range has an adjustment distance of 65 μm to 250 μm. In other words, the second focus adjusting device can be formed to adjust the focus point in a range, which is at least 65 μm or maximally 250 μm large.

In a further embodiment the second speed includes 400 mm/s to 2000 mm/s, in particular 600 to 1200 mm/s. This means that the second focus adjusting device can move the focus point with a speed, which is between 3 mm/s and 850 mm/s, in depth direction.

In a further embodiment a resolution of the second focus adjusting device has 10 nm to 250 nm, preferably 250 nm. This means that the second focus adjusting device can set the focus point accurately up to 250 nm, preferably up to 10 nm. Therein, response times can for example be in a range from 0.5 ms to 4 ms, in particular at 4 ms.

Furthermore, the treatment apparatus can comprise a control device, which is formed to control the focus adjusting devices. Thereto, the control device can comprise a computing unit for electronic data processing such as for example a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which at least the focus adjusting devices are encoded. The program code can for example also include control data for the respective laser. The control device can be formed as a control chip or control unit. The control device can for example be encompassed by a computer or computer cluster.

The ophthalmological laser of the treatment apparatus can be formed to at least partially separate a predefined corneal volume with predefined interfaces of a human or animal eye by means of optical breakthrough, in particular at least partially separate it by means of photodisruption and/or to ablate corneal layers by means of (photo)ablation and/or to effect a laser-induced refractive index change in the cornea and/or the eye lens and/or to increase a crosslinking of the cornea.

For example, the laser can be suitable to emit laser pulses in a wavelength range between 300 nm and 1400 nm, preferably between 900 nm and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The use of such lasers in the method according to the invention additionally has the advantage that the irradiation of the cornea does not have to be effected in a wavelength range below 300 nm. This range is subsumed by the term “deep ultraviolet” in the laser technology. Thereby, it is advantageously avoided that an unintended damage to the cornea is effected by these very short-wavelength and high-energy beams. Photodisruptive and/or ablative lasers of the type used here usually input pulsed laser radiation with a pulse duration between 1 fs and 1 ns into the corneal tissue. Thereby, the power density of the respective laser pulse required for the optical breakthrough can be spatially narrowly limited such that a high incision accuracy is allowed in the generation of the interfaces. In particular, the range between 700 nm and 780 nm can also be selected as the wavelength range.

Further features and advantages of one of the described aspects of the invention can result from the embodiments of another one of the aspects of the invention. Thus, the features of the embodiments of the invention can be present in any combination with each other if they have not been explicitly described as mutually exclusive.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, configurations are also to be regarded as encompassed and disclosed by the invention, which are not explicitly shown or explained in the figures, but arise from and can be generated by separated feature combinations from the execution examples and/or embodiments. Thus, configurations are also to be regarded as disclosed, which do not comprise all of the features of an originally formulated claim or extend beyond or deviate from the feature combinations set forth in the relations of the claims. To the execution examples, there shows:

FIG. 1 depicts a schematic representation of a treatment apparatus according to an exemplary embodiment.

FIG. 2 depicts a schematic representation of a treatment apparatus according to a further exemplary embodiment.

FIG. 3 depicts a schematic representation of a treatment apparatus according to a further exemplary embodiment.

In the figures, identical or functionally identical elements are provided with the same reference characters.

### DETAILED DESCRIPTION

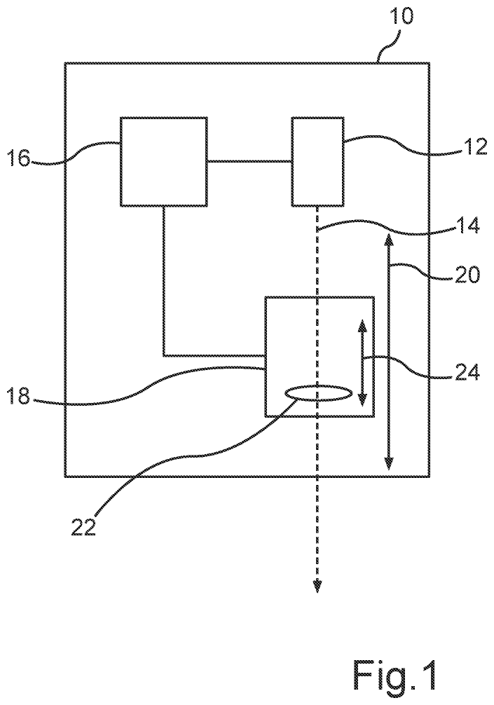

FIG. 1 shows a schematic representation of a treatment apparatus 10 with an ophthalmological laser 12, which can for example be formed for removing a tissue from a human or animal cornea by means of photodisruption and/or photoablation. Alternatively or additionally, the laser can be formed for generating a laser-induced refractive index change or crosslinking. Furthermore, the treatment apparatus 10 can comprise a control device 16, which is formed to control the ophthalmological laser 12. For example, the control device 16 can comprise a storage device (not shown) for at least temporary storage of at least one control dataset, wherein the control dataset or datasets can include control data for positioning and/or focusing individual laser pulses in the eye, in particular in the cornea.

The illustrated laser 12 can preferably be a photodisruptive and/or photoablative laser, which is formed to emit a laser beam 14 including laser pulses in a wavelength range between 300 nm and 1400 nm, preferably between 700 nm and 1200 nm, at a respective pulse duration between 1 fs and one ns, preferably between 10 fs and 10 ps, and a repetition frequency of greater than 10 kHz, preferably between 100 kHz and 100 MHz.

Furthermore, the treatment apparatus 10 can comprise further components, which are not shown here for reasons of clarity. In particular, a beam deflection device (not illustrated) can be provided, such as for example a rotation scanner, which can deflect laser pulses of the laser beam 14 in x- and y-positions.

Furthermore, FIG. 1 shows that the treatment apparatus 10 comprises at least two focus adjusting devices 18, 22, which are formed to adjust a focus point of the laser beam 14 in depth direction. This means that a z-position of the focus can be set by the focus adjusting devices 18, 22, wherein the focus adjusting devices 18, 22 can for example also be controlled by the control device 16.

In the form of configuration illustrated here, a first focus adjusting device 18 is illustrated, which can be adjusted over a first depth distance range 20, and a second focus adjusting device 22 integrated therein, which can be adjusted over a second depth distance range 24. Herein, the second focus adjusting device 22 can be formed as a lens or lens system, for example similar to an objective, wherein the lens can for example be shifted over the depth distance range 24 by an electric motor to change the focus point.

The first focus adjusting device 18, in which the second focus adjusting device 22 is integrated, can be formed as a type of stage or carrier device in this configuration, wherein the entire arrangement of the focus adjusting devices 18, 22 can thus be shifted and can change the focus point.

Therein, the first depth distance range 20 can preferably be larger than the second depth distance range 24. Particularly preferably, a speed, with which the focus point can be adjusted by the respective focus adjusting device 18, 22, is also different in both focus adjusting devices 18, 22. In particular, it is provided that a first speed of the first focus adjusting device 18 is slower than a second speed of the second focus adjusting device 22. Thus, the first focus adjusting device 18 has much stroke, but is slow, and the second focus adjusting device 22 has little stroke, but is fast. Thereby, the first focus adjusting device 18 can in particular have an inferior resolution compared to the second focus adjusting device 22.

By this arrangement, it can be achieved that a coarse setting is provided by the first focus adjusting device 18 and a fine setting of the focus point is then performed by the second focus adjusting device 22.

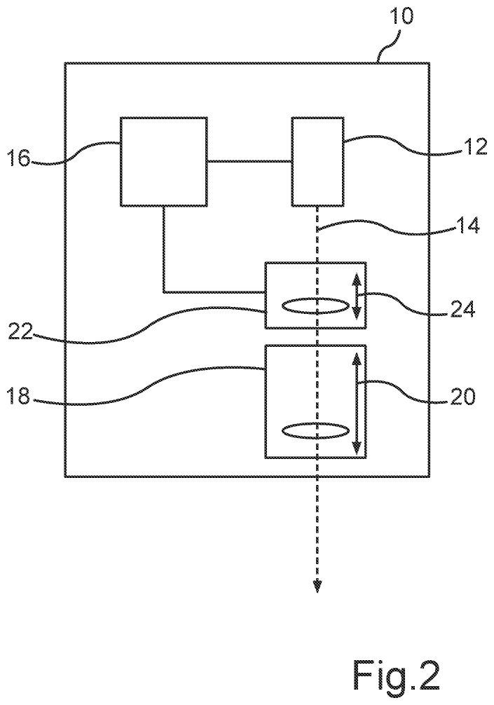

In FIG. 2, a further configuration of the treatment apparatus 10 is illustrated, which includes the ophthalmological laser 12 and the control device 16. In this configuration, the first focus adjusting device 18 and the second focus adjusting device 22 are arranged in series. Therein, the focus adjusting devices 18, 22 can again be controlled by the control device 16 for adjusting the focus point, wherein the first focus adjusting device 18 can be adjusted over the first depth distance range 20 and the second focus adjusting device 22 over the second depth distance range 24 here too, wherein the first depth distance range 20 is larger than the second depth distance range 24. Preferably, the first speed of the first focus adjusting device 18 can also be smaller than the second speed of the second focus adjusting device 22.

In this configuration, both focus adjusting devices 18, 22 can comprise one or more lenses, which are formed movable along the laser propagation direction of the laser beam 14. Thus, a focus point can be shifted in the depth by suitable setting of the respective focus adjusting device 18, 22 here too, wherein a coarse setting can thus again be performed by the first focus adjusting device 18 and a fine setting by the second focus adjusting device 22.

In this representation, the second focus adjusting device 22 is shown in front of the first focus adjusting device 18 based on the laser 12, wherein this representation is only exemplary and the order can also be interchanged.

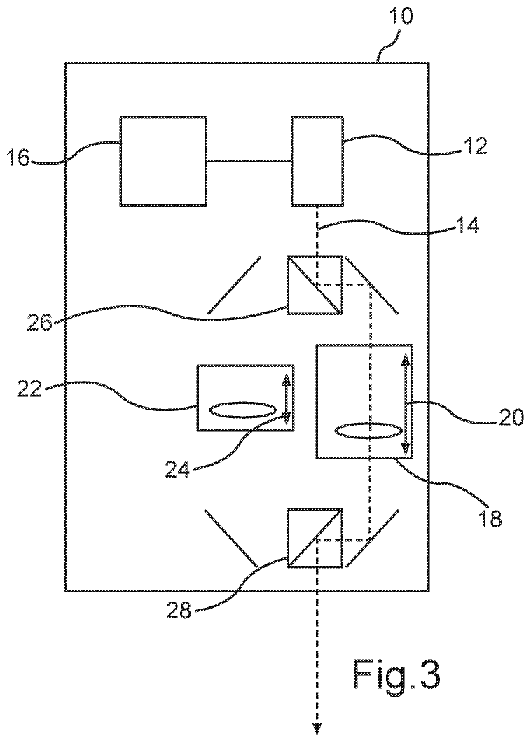

In FIG. 3, a further exemplary representation of a configuration of the treatment apparatus 10, which includes the ophthalmological laser 12 and the control device 16, is shown. In this configuration, the first focus adjusting device 18 and the second focus adjusting device 22 can be arranged in parallel with each other, which means that the laser beam 14 can be selectively directed via one of the two focus adjusting devices 18, 22. Here too, in the same manner as in the previously shown configurations, the first depth distance range 20 can be larger than the second depth distance range 24 and the first speed can be smaller than the second speed.

In order to selectively direct the laser beam 14 via one of the two focus adjusting devices 18, 22, a switching element 26 can furthermore be provided, for example an adjustable mirror, which can deflect the laser beam into the respective optical path of the focus adjusting device 18, 22. Herein, the switching element 26 as well as the focus adjusting devices 18, 22 can again be controlled by the control device 16.

After passage through the respective focus adjusting device 18, 22 with the desired focus setting, a further switching element 28 can preferably be provided, which again converges the laser beam 14 in a starting position. This means that no matter whether the laser beam is directed through the first focus adjusting device 18 or the second focus adjusting device 22, the laser beam 14 again exits the treatment apparatus 10 at the same location.

In all of the previously shown configurations, it can preferably be provided that the first depth distance range 20 has an adjustment distance of 500 μm to 5 mm, preferably of 2 to 3 mm. Therein, the first speed of the first focus adjusting device 18 can be between 3 mm/s and 850 mm/s, in particular between 50 and 100 mm/s. Furthermore, a resolution of the first focus adjusting device 18 can have 100 nm to 1.5 μm, preferably 400 nm. Herein, it can be provided that response times of the first focus adjusting device 18 have 1 ms to 400 ms, in particular 15 to 30 ms.

The second depth distance range 24 of the second focus adjusting device 22 can preferably have an adjustment distance of 65 μm to 250 μm. Furthermore, the second speed of the second focus adjusting device 22 can include 400 mm/s to 2000 mm/s, in particular 600 to 1200 mm/s. Furthermore, a resolution of the second focus adjusting device 22 can preferably have 10 nm to 250 nm, preferably 250 nm, wherein response times can for example include 0.5 ms to 4 ms.

Overall, the examples show, how a treatment apparatus with multiple scanners adjustable in z-direction can be provided, in particular to generate more complicated and asymmetric, respectively, treatment patterns in improved manner.

1. A treatment apparatus with at least one ophthalmological laser, wherein the treatment apparatus includes at least two focus adjusting devices, which are formed to adjust a focus position of a laser beam of the laser in a depth direction, wherein the first focus adjusting device is formed to adjust the focus point over a first depth distance range with a first speed, and wherein the second focus adjusting device is formed to adjust the focus point over a second depth distance range with a second speed, wherein the first depth distance range is larger than the second depth distance range and the first speed is smaller than the second speed.

2. The treatment apparatus according to claim 1, wherein the second focus adjusting device is integrated in the first focus adjusting device, and wherein the first focus adjusting device is formed to also adapt the focus point of the second focus adjusting device upon adjusting the first focus adjusting device in depth direction.

3. The treatment apparatus according to claim 1, wherein the first and the second focus adjusting device are arranged in series.

4. The treatment apparatus according to claim 1, wherein the first and the second focus adjusting device are arranged in parallel in the treatment apparatus, wherein the treatment apparatus comprises a switching element, which is formed to selectively adjust the laser beam via the first or the second focus adjusting device.

5. The treatment apparatus according to claim 1, wherein the first depth distance range has an adjustment distance of 500 μm to 5 mm, in particular of 2 to 3 mm.

6. The treatment apparatus according to claim 1, wherein the first speed includes 3 mm/s to 850 mm/s, in particular 50 to 100 mm/s.

7. The treatment apparatus according to claim 1, wherein a resolution of the first focus adjusting device has 100 nm to 1.5 μm, in particular 400 nm.

8. The treatment apparatus according to claim 1, wherein the second depth distance range has an adjustment distance of 65 μm to 250 μm.

9. The treatment apparatus according to claim 1, wherein the second speed includes 400 mm/s to 2000 mm/s, in particular 600 to 1200 mm/s.

10. The treatment apparatus according to claim 1, wherein a resolution of the second focus adjusting device has 10 nm to 250 nm, in particular 250 nm.
